(19) United States
(12) Patent Application Publication
(10) Pub. No.: US 2010/0318017 A1
(43) Pub. Date: Dec. 16, 2010

(54) Method of improving vision using contact lenses and corneal collagen crosslinking

(76) Inventors: Scott E. Lewis, Ponte Vedra, FL (US); Jim Edwards, Lawton, OK (US); Cynthia Roberts, Columbus, OH (US); Aylin Kilic, Kavaklidere-Ankara (TR)

Correspondence Address: ROGERS TOWERS, P.A., 1301 RIVERPLACE BOULEVARD, SUITE 1500, JACKSONVILLE, FL, 32207, US

(21) Appl. No.: 12/456,038

(22) Filed: Jun. 11, 2009

(51) Int. Cl. A61F 9/00; A61B 3/107; A61N 5/00; G02C 7/04

(52) U.S. Cl. 604/20; 351/247; 351/160 R

(57) ABSTRACT

A method of improving vision by reshaping the cornea to correct for abnormal surface curvature by measuring the surface topography of the cornea, providing a rigid contact lens to reshape the cornea into a more desirable configuration, wearing the contact lens for sufficient time to achieve the reshaping, removing the contact lens, applying an alcohol solution to the epithelial layer, applying a riboflavin solution to the cornea, and subjecting the cornea to UV light for sufficient time to induce cross-linking of collagen fibers within the cornea, whereby the cornea is stabilized in the reshaped desirable configuration.

This application claims the benefit of U.S. Provisional Patent Application Ser. No. 61/131,583, filed Jun. 6, 2008.

### BACKGROUND OF THE INVENTION

This invention relates generally to treatment methodologies in the field of vision improvement for conditions where the cornea has adopted an abnormal shape. More particularly, the invention relates to methodologies incorporating corneal collagen crosslinking.

Certain vision problems are the result of changes in the shape of the cornea. Aging, disease or injury may result in the weakening of the cornea, such that the normal curvature of the cornea is replaced by localized bulging or distortion. The abnormal curvature of the cornea may change the refractive error, resulting in moderate or severe blurriness or astigmatism. Keratoconus and corneal ectasia are two common conditions that result in this abnormal curvature of the cornea. Surgical solutions to this problem include corneal transplant PKP or the insertion of small semi-circular plastic rings to increase the structural integrity of the cornea.

A relatively recent treatment methodology for abnormal curvature problems of the cornea due to keratoconus or the like is known as corneal collagen crosslinking with riboflavin (C3-R). The C3-R method involves the application of a riboflavin solution to the cornea, with or without removal of the superficial epithelial barrier on the surface of the cornea, followed by exposure to UV light. Removal of the epithelial barrier is accomplished by exposure to alcohol and then physical scraping of the corneal surface. It is thought that removal of the epithelial layer optimizes the treatment, since the epithelial layer interferes to some degree with riboflavin and/or UV light penetration in the stroma. The UV light exposure in the presence of riboflavin triggers crosslinking of the collagen fibers in the cornea. The crosslinked collagen create natural anchors to stabilize the cornea, thereby arresting the further distortion of the cornea.

A problem with the known C3-R treatment methods is that the treatment merely prevents further distortion of the cornea. The crosslinked collagen fibers stabilize the cornea in the abnormal configuration. Thus, the C3-R treatment as currently practiced does not correct vision problems that are present when the treatment is administered, as there is no reshaping of the cornea.

It is an object of this invention to provide an improved C3-R treatment methodology that addresses the shortcomings of the current C3-R methodology, in that in addition to stabilizing the cornea by increasing its structural integrity, the abnormal curvature or distortion of the cornea resulting in vision problems is corrected as well. It is an object of this invention to provide an improved C3-R treatment that is applicable to a broad spectrum of vision problems, including near-sightedness, far-sightedness and astigmatism, wherein refractive correction is needed due to distortion in the curvature of the cornea.

### SUMMARY OF THE INVENTION

The invention is a treatment methodology for vision problems resulting from abnormal curvature of the cornea, the methodology comprising a combination of the steps of acquiring topographical measurements of the corneal surface, determining the parameters for a customized rigid gas-permeable contact lens necessary to reshape the corneal surface into a more desirable curvature, wearing the customized rigid RGP contact lens for a period of time such that pressure from the lens reshapes the cornea, removing the lens and applying alcohol to loosen the epithelial layer over the cornea, applying riboflavin solution to the cornea, and exposing the cornea to UV light to induce crosslinking of the collagen fibers. In this manner, the crosslinked collagen fibers stabilize the cornea in the corrected curvature such that post-treatment vision is improved.

### BRIEF DESCRIPTION OF THE DRAWINGS

FIG. 1 is a flow chart illustrating the methodology.

FIG. 2 is an illustration showing the rigid contact lens in position on a cornea having an abnormal surface curvature.

FIG. 3 is an illustration showing the reshaped cornea wherein the abnormal surface curvature has been removed.

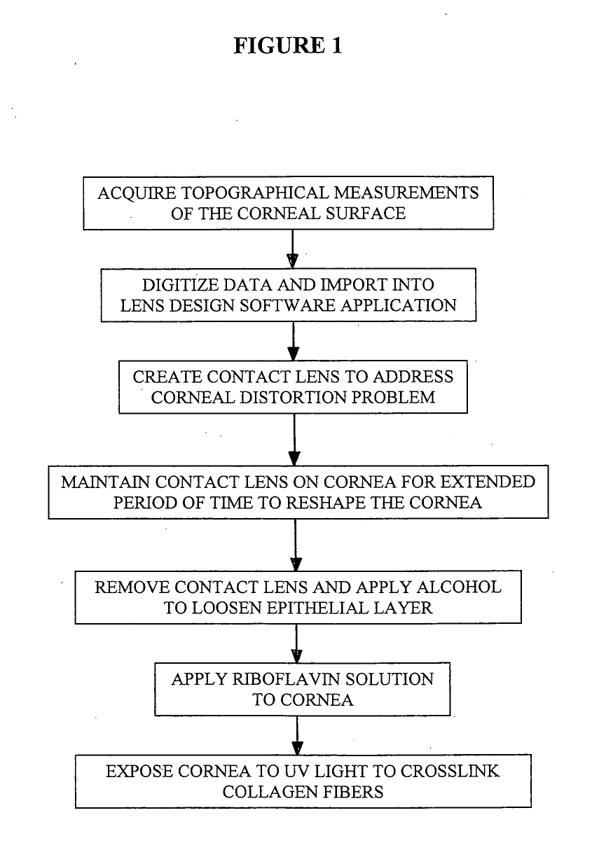

### DETAILED DESCRIPTION OF THE INVENTION

In general, the invention is a treatment methodology to address vision problems resulting from abnormal curvature or distortion of the corneal surface, such as may result for example from aging, diseases or injury. The methodology is particularly applicable to the treatment of vision problems resulting from keratoconus, corneal ectasia or the like. The treatment addresses near-sightedness, far-sightedness and astigmatisms, and may be also used for example to address pellucids, dystrophy and corneal erosions.

The initial step involves taking topographical measurements or mapping the cornea surface 11 in order to determine the three-dimensional parameters of any bulge, protrusion or other abnormal curvature 12 of the corneal surface. Various topography apparatuses capable of taking the measurements are well known in the field, such as those sold under the brand names SCOUT or MEDMONT for example. The equipment utilizes sophisticated software to produce data and images corresponding to the true topography of the cornea. After filtering or correcting the data set for small irregularities, the data is digitized and imported into a rigid contact lens CAD/CAM software design application to calculate the lens parameters necessary to reshape the cornea surface 11 into a desired configuration. The corrected configuration is chosen to address the vision problems resulting from the corneal distortion, such that the refractive error will be reduced due to the cornea assuming a more correct configuration.

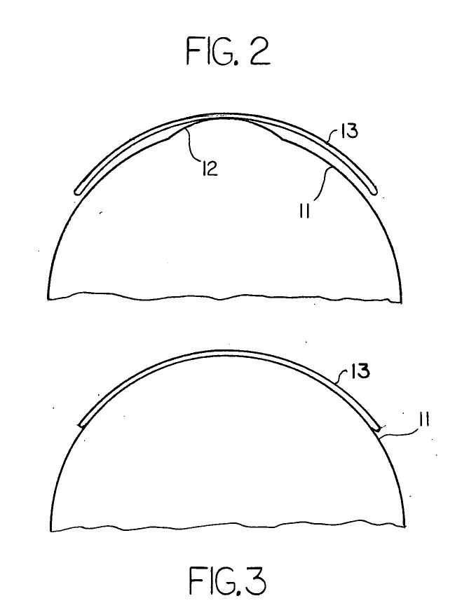

The lens parameters are used to create a rigid gas-permeable contact lens 13 that will reshape the cornea surface 11 by applying pressure against the outwardly distorted portions 12 of the cornea, such as for example the bulge produced by keratoconus. The lens 13 is configured to contact the cornea at the steepest region of the distortion, as shown in FIG. 2. In the region peripheral to this point or area of maximum distortion, the lens 13 will not contact the cornea surface 11, such that a partial or fully annular gap is present around the bulge 12. This gap allows the cornea surface 11 to adapt into the desired curvature defined by the contact lens 13 as the area of maximum distortion is reduced by pressure from the rigid contact lens 13. The rigid contact lens 13 is worn continuously for a time period sufficient to reshape the cornea into the corrected configuration, as shown in FIG. 3, and may require wearing the lens 13 for approximately one week. After this period of time, the lens 13 is removed. The result at this point is that the cornea surface 11 has now been temporarily reshaped into a more desirable configuration, thereby correcting or improving the vision problems caused by the surface distortions 12 that were present before application of the contact lens.

An alcohol solution is then applied to the surface of the eye in order to loosen the epithelium over the cornea. The loosened epithelial layer may be removed by scraping, but this is not necessary. A riboflavin solution is then applied to the cornea and the eye is exposed to UV light for a sufficient period to initiate cross-linking of the collagen fibers within the cornea. A thirty minute UV exposure has been shown to be suitable to accomplish sufficient cross-linking to stabilize the cornea. The influence of the contact lens design results in increased UV absorption in the thinnest areas of the cornea and decreased absorption in the thicker areas. Because the cornea has been reshaped into a more desirable configuration due to the extended wearing of the rigid contact lens 13 when the cross-linking is induced, the stabilized shape of the cornea after the treatment results in improved vision.

We claim:

1. A method of improving vision by reshaping the cornea comprising the steps of:
measuring the surface topography of the cornea,
providing a rigid contact lens to reshape the cornea into a desired configuration,
wearing the contact lens for sufficient time to achieve reshaping of the cornea,
removing the contact lens,
applying a riboflavin solution to the cornea, and
subjecting the cornea to UV light for sufficient time to induce cross-linking of collagen fibers within the cornea,
whereby the cornea is stabilized in the desired configuration.

2. The method of claim 1, further comprising the step of applying an alcohol solution to the cornea surface to loosen the epithelium after the contact lens is removed and prior to applying the riboflavin solution.

3. The method of claim 3, further comprising the step of removing the loosened epithelium.

4. A method of improving vision by reshaping the cornea into a desired configuration comprising the steps of:
mapping the surface topography of the cornea to determine abnormal surface curvature,
calculating the physical parameters for a rigid contact lens to reshape the cornea into a desired configuration for improved vision,
providing a rigid contact lens to reshape the cornea into the desired configuration, said rigid contact lens configured to correct the abnormal surface curvature determined in the cornea,
wearing the contact lens for sufficient time to achieve reshaping of the cornea into the desired configuration,
removing the contact lens,
applying a riboflavin solution to the epithelial layer of the cornea, and
subjecting the cornea to UV light for sufficient time to induce cross-linking of collagen fibers within the cornea,
whereby the cornea is stabilized in the desired configuration.

5. The method of claim 4, further comprising the step of applying an alcohol solution to the cornea surface to loosen the epithelial layer after the contact lens is removed and prior to applying the riboflavin solution.

6. The method of claim 5, further comprising the step of removing the loosened epithelial layer.
